(19) United States
(12) Patent Application Publication
(10) Pub. No.: US 2020/0017910 A1
(43) Pub. Date: Jan. 16, 2020

(54) Method of creating an epigenetic skin profile associated with skin quality

(71) Applicants: Weiwei Li, Woodbury, NY (US); Jessica Mliu Li, Woodbury, NY (US); William Lei Lee, Mineola, NY (US)

(72) Inventors: Weiwei Li, Woodbury, NY (US); Jessica Mliu Li, Woodbury, NY (US); William Lei Lee, Mineola, NY (US)

(21) Appl. No.: 15/998,159

(22) Filed: Jul. 10, 2018

(51) Int. Cl. C12Q 1/6881; C12Q 1/686; C12Q 1/6806

(52) U.S. Cl. CPC C12Q 1/6881; C12Q 1/686; C12Q 2600/16; C12Q 2600/166; C12Q 2600/154; C12Q 1/6806

(57) ABSTRACT

This invention is related to a method useful for creating an epigenetic skin profile associated with skin quality in a biological sample through DNA methylation identification and analysis of the specific gene markers.

### CROSS-REFERENCE TO RELATED APPLICATIONS

Not applicable

### STATEMENT REGARDING FEDERALLY SPONSORED RESEARCH OR DEVELOPMENT

Not applicable

### REFERENCE TO A MICROFICHE APPENDIX

Not applicable

### BACKGROUND OF THE INVENTION

### Field of the Invention

This invention is related to a method, useful for creating an epigenetic skin profile associated with skin quality in a biological sample.

### Description of the Related Art

The skincare marketplace offers a very large, often confusing, selection of products. Despite many of these products claiming to have scientific substance to mechanisms (e.g., anti-aging, skin brightening, moisture retention, wrinkle firming, etc), consumer buying decisions often depend on non-scientific factors such as flashy advertising, brand loyalty, referrals, social media influencers, and consumer reviews. However, there is always an inherent doubt by the consumer about whether the selected product is working as claimed, fueling a constant search for the ideal product that aligns with his or her skin features. Such search is made difficult due to: (a) lack of transparency in scientific information and standards; (b) consumer uncertainty on whether he or she falls within the segment the product is developed for; and (c) the unique and dynamic aspects of one's skin. An unintentionally mismatched product selected by the consumer may adversely damage one's skin, and thus consumers are increasingly willing to pay a small premium for personalized solutions.

The skin quality and/or beauty is reflected by the skin appearance but mainly determined by the following factors: aging, firmness/elasticity, moisture, DNA damage and repair, skin cell renewal/regeneration capacity, oxidant and antioxidant capacity, sensitivity and inflammatory control, and skin pigmentation. It was recognized that personalized skincare needs to first obtain useful information regarding skin quality and/or beauty or to properly characterize one's skin status. However very few scientific methods are currently established to achieve such a goal.

One of these methods is image-based artificial intelligence or machine learning algorithms analysis of skin quality and/or beauty. However, these algorithms utilize superficial data solely from photos and surveys, which are unable to serve as reliable machine learning sources due to the lack of correlation with actual scientific data. In the end, without such scientific basis, they function no differently than an in-person consultant relying on holistic guesswork approaches.

Another one of these methods is genetic testing. These tests are based on single nucleotide polymorphism (SNP) markers [Naval J et al: Clin Cosmet lnvestig Dermatol, 2014; Harper, R et al: WO2014176425A1. 2014], which only reveal intrinsic risks or susceptibility to skin diseases in a small and specific population. This limiting factor is due to genetics being established at birth and is neither changeable nor reversible in one's entire life. Additionally, these personal genomic tests use saliva samples as the input, which cannot reflect the current state of one's skin quality. Thus, genetic testing is not able to determine and monitor dynamic changes of the skin from aging or environmental exposure, and consequently is not able to truly identify dynamic change of skin quality.

Because of the lack of useful personalized skin quality and/or beauty data, skincare product selections and purchases made by consumers are still mostly guesswork. In recent years, substantial evidence shows that epigenetic switches are dynamic and reversible and can be regulated by the intervention of medicine, lifestyle, and environmental factors [Tompkins J D et al: Proc Natl Acad Sci USA. 109, 2012, Burggren W W: Journal of Experimental Biology, 218, 2015]. Thus epigenetics would most accurately reflect skin quality and its dynamic change in response to environmental stimuli. Epigenetics is a naturally occurring biological mechanism that primarily refers to the modification of DNA (i.e., DNA methylation). DNA methylation occurs by the covalent addition of a methyl group (CH₃) at the 5-carbon of the cytosine ring, resulting in 5-methylcytosine (5-mC). DNA methylation is essential in regulating gene expression in nearly all biological processes including development, growth, and differentiation (Laird P W et al: Annu. Rev. Genet. 30, 1996; Reik W et al: Science, 293, 2001). Alterations in DNA methylation have been demonstrated to cause a change in gene expression. For example, hypermethylation leads to gene silencing or decreased gene expression, while hypomethylation activates the genes or increases gene expression. Thus gene/region-specific or genome-wide analysis of DNA methylation or 5-methylcytosine (5-mC) could exploit DNA methylation, the most well characterized epigenetic process, as a detection basis for dynamic skin quality and appearance changes.

Each person's epigenetic makeup is unique and can be potentially heritable, but more importantly, biological aging, diet, disease, exercise, and environmental exposure can all cause chemical modifications around the genes that will turn those genes on or off over time. Thus, DNA methylation status from a skin sample itself would be a real indicator of skin quality/beauty uniqueness and dynamics. Several studies were done to show that reduced methylation of some of genes is associated with skin aging, and sun exposure can increase skin aging through changes in methylation levels of a group of genes [Bormann F et al: Aging Cell, 15, 2016; Gronniger E et al: PLoS Genet, 6, 2010]. However, currently there are no epigenetic profiles associated with skin quality and/or beauty to be available. Further there is still ample need to generate such epigenetic skin profiles that would be truly associated with skin quality and/or beauty and dynamic change, and that can be used for personalized skincare.

### BRIEF SUMMARY OF THE INVENTION

The present invention provides the methods for generating an epigenetic skin profile associated with skin quality and/or beauty comprising:

1) collecting a biological sample from a skin site in a non-invasive manner including lifttape, cotton tip swab, and adhesive sheet;
2) determining epigenetic status of the said biological samples with an appropriate method including methylation-specific PCR and DNA sequencing, more preferably targeted methylation-sequencing;
3) identifying and analyzing epigenetic markers that are associated with skin quality and/beauty, thereby generating the individualized epigenetic profile that reveals and gives an overall rating of the current status of one or more indicators of skin quality and/or beauty.

Thus the invention allows a DNA methylation skin profile associated with skin quality and/or beauty to be achieved. The invention is based on the finding that the methylation status in a panel of genes affects functional change of one or more indicators of skin quality and/or beauty. The invention is also based on the finding that the methylation grade of the same panel of genes is proportional to the dynamic change of one or more indicators of skin quality and/or beauty. Therefore the method presented in this invention significantly overcomes the weaknesses existing in the prior technologies and enables a DNA methylation skin profile associated with skin quality and/or beauty to be determined and analyzed efficiently.

### BRIEF DESCRIPTION OF THE DRAWINGS

FIG. 1. shows a diagram of the process for generating an epigenetic skin profile associated with skin quality and/or beauty. The process involves: (1) skin sample collection with adhesive tape; (2) DNA isolation and modification; (3) modified DNA amplification; (4) targeted amplicon library construction; (5) deep sequencing; (6) bioinformatics analysis of the deep sequencing data; (7) overall rating of the current status of one or more indicators of skin quality and/or beauty.

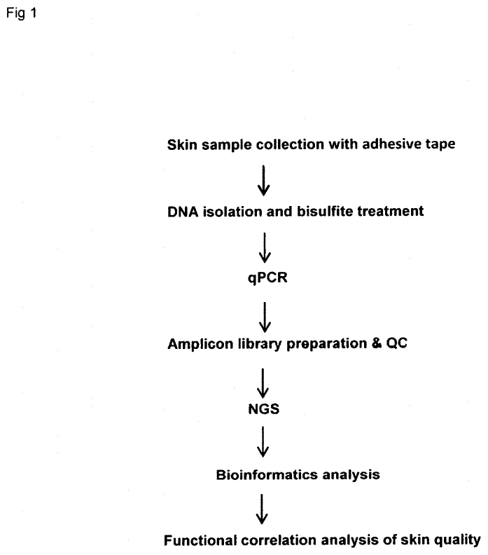

FIG. 2. shows the DNA yield obtained from the different skin samples.

FIG. 3. shows multiplexed qPCR amplification results of the modified DNA from different skin samples. 4-6 primer pairs are multiplexed in each well and a total of 20 primer pairs targeting 20 different gene markers are used for each sample within 4 wells. Water is added as a negative control and fully methylated HeLa DNA is used as a positive control. Cts obtained for the multiplexed gene markers range from 24 to 36.

FIG. 4. shows bioanalyzer results of library DNA after PCR amplification.

FIG. 5. shows bioinformatics analysis of a panel of DNA methylation makers.

FIG. 6. shows that the DNA methylation grade of one of the gene markers ELOVL2 is associated with change in skin aging, one of the indicators of skin quality and/or beauty. DNA methylation grade for the gene was calculated based on the ratio of the unconverted cytosine at CpG sites to the total CpG sites of the detected DNA sequences. Aging score is calculated based on the comparison of the marker methylation grade of the sample to correlation curves of ELOVL2 methylation markers versus chronological age previously reported.

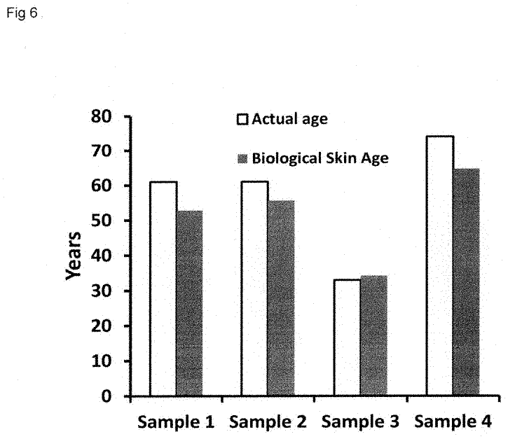

FIG. 7. shows the DNA methylation level of a panel of markers is associated with other skin quality and/or beauty indicators. These gene markers include AQP3 for moisture, XPC and ERCC1 for DNA repair, c-EBP-beta for skin renewal capacity, and SOD2, GPX1 and NQO1 for antioxidant capacity.

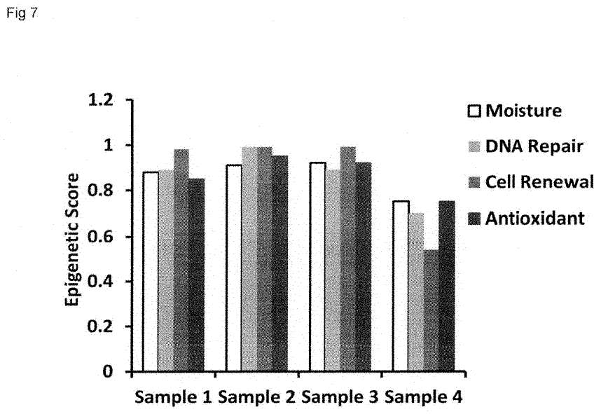

### DETAILED DESCRIPTION OF THE INVENTION

According to the method of this invention a non-invasive manner is used for the collection of skin cell samples. The manner involves the use of a cotton tip swab, or an adhesive tape. For using cotton tip swab, the swab is rolled over the surface of the skin using moderate pressure and circular motions. Rotating the swab on its long axis ensures maximum contact between the swab and the skin. The swabbed areas include ear, nose, face and hands with 7-8 times for each area in order to ensure that a sufficient number of skin cells are collected. Various cotton tip swabs can be commercially available. For using adhesive tape, place the adhesive tape discs on the selected skin areas including face and hands. One area is applied each time. Hold and press the tape for approximately 2 seconds each time and then detached for several times. The adhesive tape may include but is not limited to various adhesive tapes for medical or dermatological purpose consist of acrylic adhesives, polyesters, polyurethanes, silicone copolymers, polyisobutylenes, and ethylene vinyl acetates.

The skin cell samples collected on the cotton swab or adhesive tape will be used for DNA isolation. There are many methods used for DNA isolation from various samples, However for this kind of sample which is collected on swab or adhesive tape, we prefer to use the following method to isolate DNA from a skin sample collected on the adhesive tape: Place the tape into the tube containing sample lysis buffer, submerge the sample for 5 min and then wash the surface with the collected sample with lysis buffer for 7-8 times. The tape is then removed from the tube and protein digestion enzyme solution is added and incubated with the solution containing the sample at an appropriate temperature for degrading protein and releasing DNA. DNA is then purified and collected by using DNA affinity magnetic beads.

In an embodiment of the invention, the purified DNA sample is treated with bisulfite reagents. These bisulfite reagents may include but are not limited to sodium bisulfite, potassium bisulfite, ammonium bisulfite, magnesium bisulfite, sodium metabisulfite, potassium metabisulfite, ammonium metabisulfite and magnesium metabisulfite. Bisulfite salts such as sodium bisulfite or ammonium bisulfite can convert cytosine to uracil and leave the methylated cytosine (5-mC) unaltered. Thus after bisulfite treatment 5-mC in the DNA remains the same and unmodified cytosines will be changed to uracil. The bisulfite treatment can be performed by using the methods disclosed in prior art or commercial kits such Bisulflash DNA modification kit (Epigentek) or Imprint DNA modification kit (Sigma) with appropriate modification of the bisulfite treatment process. For achieving optimal bisulfite conversion efficiency, the bisulfite reaction should be carried out in an appropriate concentration of bisulfite reagents, appropriate temperature and appropriate reaction time period. A reagent such as potassium chloride that reduces thermophilic DNA degradation could be also used in bisulfite treatment so that DNA bisulfite process can be much shorter without interrupting a completed conversion of unmethylated cytosine to uracil and without a significant thermodegradation of DNA resulted from depurination.

Once DNA bisulfite conversion is complete, nucleic acids are captured, desulphonated and cleaned. The bisulfite-treated DNA can be captured by a solid matrix selected from silica salt, silica dioxide, silica polymers, magnetic beads, glass fiber, celite diatoms and nitrocellulose in the presence of high concentrations of chaotropic or non-chaotropic salts. The bisulfite-treated DNA is further desulphonated with an alkalized solution, preferably sodium hydroxide at concentrations from 10 mM to 300 mM. The DNA or RNA is then eluted and collected into a capped microcentrifuge tube. An elution solution could be DEPC-treated water or TE buffer (10 mM Tris-HCL, pH 8.0 and 1 mM EDTA).

According to the method of this invention, such bisulfite-converted DNA can be used for skin quality and/or beauty indicator-associated gene marker methylation/demethylation determination. A panel of genes whose methylation or demethylation is used for biomarkers of skin quality and/or beauty indicators is comprised of the genes that are responsible for the indicators such as aging, firmness and elasticity, moisture, DNA damage and repair, skin cell renewal/regeneration capacity, oxidation and antioxidant capacity, sensitivity and inflammatory response and skin pigmentation.

Aging: Age-dependent epigenetic change (DNA methylation signature) has become a hallmark of aging. Skin aging shows skin dryness, loss of firmness, decrease in elasticity, and increased risk of wrinkles, acne, and cancer. Firmness and Elasticity: Collagen and elastin proteins hold your skin together and maintain elasticity. Decrease of these proteins causes fine wrinkles, elasticity loss, reduced thickness, and skin looseness. The major enzymes or proteins which are responsible for formation, metabolism, secretion and glycation of collagen and elastin are epigenetically controlled and regulated through DNA methylation or histone modification. Moisture: It is maintained by water transportation proteins and is essential for smooth and bright skin. It also helps with the elasticity of stratum corneum, preventing tightness and cracking. Activation or repression of water transportation proteins is critically dependent on epigenetic regulation. DNA Damage/Repair: Environmental-induced DNA damage is mainly due to excessive exposure to UV radiation in the form of sunlight and cigarette smoke as well as pollutants. Epigenetic activation of DNA repair genes or repression of DNA damage reaction pathways become critical for control DNA damage/repair and cell regeneration. Cell regeneration: Skin cell renewal/regeneration refers the regrowth of lost skin cells in response to damage or cell death. Attenuated ability of cell renewal/regeneration will cause aged skin with wrinkles, age spots and dryness. Your skin also becomes thinner and loses fat, making it less plump and smooth. Epigenetic changes are proven to be critical determinants for control/regulation of cell renewal and regeneration. Sensitivity Response: Sensitive skin and inflammatory response from an immunity reaction to environmental stimuli such as irritants or pollutants can lead to itchiness, redness, peeling, etc. and are often due to action of inflammatory protein such as cytokines. Regulatory gene/protein/enzymes for sensitive and inflammatory response pathways are critically controlled by DNA/histone modification-related gene activation/inactivation. Oxidation/Anti-oxidation: Oxidative damage results from an imbalance between free radical generation and antioxidant defenses, with unfavorable symptoms including age spots, wrinkles, and sagging. It is well known that the oxidation and anti-oxidant pathways are controlled by epigenetics, especially by DNA methylation/demethylation of the genes regulating these pathways. Pigmentation: Pigmentation or coloration of the skin is dependent on the amount of melanin present in the skin with over-pigmentation from sun exposure or aging leading to skin disease. Skin pigmentation is closely related to epigenetic activation or inactivation of regulatory genes of pigmentation.

Skin aging-associated gene markers mainly include but are not limited to: TOM1L1, NPTX2, ADAR, ITGA2B, PDE4C, CLCN6, TBX20, STAT5A, WT1, ASPA, KCNQ1DN, TRIM58, GRIA2, SEC3112, DDAH2, TET1, TET2, ELOVL2, p16INK4a, BIRC4BP, ITGA2B, and EDARADD. The methylation or demethylation of these gene markers is proven to be strongly associated with aging. For example, promoter methylation of ELOVL2 is increased with increased age while the promoter methylation of EDARADD is reduced with aging.

Firmness and Elasticity-associated gene markers mainly include but are not limited to: MMP1, MMP2, MMP3, MMP-8, MMP-9, MMP-13, SEC31L2, B3GALT5, RAGE, STXBP5L, COL1A1, COL1A2, COL3A1, COL4A1, COL5A1, TIMP-1, TIMP-2, P-63, Satb1, ELN, ACE, FOXO3, and HIF1A. The methylation or demethylation of these genes is closely associated with collagen and elastin protein production, secretion, and degradation. For example, demethylation of MMP1 activates MMP1 expression and function as interstitial collagenase and fibroblast collagenase, degrades collagen types I, II, and III. While RAGE methylation would reduce glycated collagen.

Moisture-associated gene markers mainly include but are not limited to: AQP-1, AQP-3, AQP5, AQP11, loricrin, involucrin (INV), filaggrin (FLG), and Brg1. Inactivation or activation of these genes through methylation regulation will cause functional protein decrease or increase. These proteins are responsible for skin hydration through water transportation and retention.

DNA Damage/Repair-associated gene markers mainly include but are not limited to: MBD4, MBD2, TDG, OGG1, MUTYH, NEIL1, NEIL2, NEIL3, MGMP, MSH2, MSH3, MSH6, MLH1, MLH3, XPA, XPC, RAD23A, RAD23b, DDB1, DDB2, CETN2, RPA1, RPA2, RPA3, ERCC1, ERCC2, ERCC3, ERCC4, ERCC5, ERCC6, ERCC8, GTF2H1, GTF2H2, GTF2H3, GTF2H4, GTF2H6, CDK7, CCNH, MNAT1, LIG1, UVSSA, XAB2, MMS19, RAD51, RAD51B, RAD51D, XRCC2, XRCC3, XRCC4, XRCC5, XRCC6, RAD50, and RAD52, The skin DNA damage mainly caused by ultraviolet and can be efficiently repaired through nucleotide excision repair (NER), which is a specialized UV-induced DNA damage repair system. Methylation-caused inactivation of DNA repair genes, especially NER genes such as ERCC2 and XPC will cause defective repair of DNA damage.

Cell regeneration/differentiation-associated gene markers mainly include but are not limited to: DDAH2, c-EBP-beta, p63, β1 integrin, K14, KMT2D, IGF-1, Dnmt1, Fra-2/AP-1, P16, P21, P27, TLR3, S1007A, Survivin, OVOL2, SOX2, CBX4, K1, K6, K10, K16, k17, CTGF, and FLG. The methylation-caused inactivation of cell regeneration or renewal genes such as P63 and c-EBP-beta will reduce skin cell regeneration ability and methylation of differentiation regulating genes such as SOX2, and CPX4, or demethylation of differentiation-marker genes such as K16 and K17 results in deregulation of these genes, which in turn cause premature skin terminal differentiation and epidermal thinning.

Sensitivity Response-associated gene markers mainly include but are not limited to: TNF-a, IL-6, SOCS1, TNFAIP3, IL-8, NF-kb, IL-1a, IL-1b, TNIP1, IL12B, IL13, CARD11, CCL5, IL-17, CCL2, INF-α, INF-β, IL-γ, IL-5, IL-18, IL-10, P38, ERK, JNK, MAP3K, CREB, SRF, TRAF3, and TLR2. The sensitivity response of skin can be caused by expression of inflammatory genes such as TNF-α and IL-18, which are activated through demethylation of these genes or due to methylation-caused inactivation of anti-inflammatory genes such as P-38 and TRAF3.

Oxidation/Anti-oxidation-associated gene markers mainly include but are not limited to: NRF2, NF-kappaB, FOXM1, NQO1, SOD2, GPX1, GPX2, GPX4, Catalase, GCLC, NADPH, TXN, G6PD, HMOX1, TXNRD1, PRDX3, PRDX4, GSTP1, SOD1, GCLM, GBA, SMPD1, and HO-1. Methylation-regulated expression of these genes, as anti-oxidative-stress system, can scavenge reactive oxygen species (ROS) that arise from chemical, physical, and metabolic challenges through glutathione system and thioredoxin system.

Skin pigmentation-associated gene markers mainly include but are not limited to: KITL, TYR, TYRP1, KRT75, SLC24A5, SLC45A2, IRF4, MC1R, ASIP, POMC, OCA2, MITF, RAB27A, and MLPH. These genes are responsible for the biochemical pathway of melanogenesis, converting the amino acids phenylalanine, tyrosine and cysteine to melanin. Methylation-regulated gene activation or inactivation will cause skin pigmentation change.

The methods for gene methylation/demethylation determination may include but are not limited to MS-PCR, Primer extension, COBRA, MethyLight, ConLight-MSP, McMSP, MSP/DHPLC, SNuPE, HM-MethyLight, strand displacement assay, ligase chain reaction, rolling circle amplification, loop-mediated amplification, Oligonucleotide microarrays, pyrosequencing, whole genome bisulfite sequencing (WGBS), reduced representative bisulfite sequencing (RRBS) and targeted bisulfite sequencing. However, a suitable method for routine use should be rapid, cost-effective enough with high sensitivity and specificity. According to this invention, the method for skin quality and/or beauty indicator-associated gene methylation/demethylation determination is related to multiplex real-time gene methylation amplification. According to this method, the CpG-rich regions in the promoter or exon 1 of a gene marker can be amplified using at least two primers and a probe which are complementary to a methylated DNA converted with bisulfite. The primers are specific to methylated DNA of interest and are able to initiate synthesis of a primer extension product. A sequence-specific probe is able to hybridize to the target between forward and reverse primer sites. The probe is labeled with a fluorescent dye at 5′ portion. The fluorescent dyes may include but are not limited to FAM, TET, Texas-red, cy3, cy5, JOE, HEX, MAX, ROX, TAMRA and VIC. The probe is quenched with a black hole quencher and conjugated with a minor groove binder (MGB) protein at its 3′ portion. The MGB is able to fold into a minor groove of DNA duplex created between the target sequence and probe. Thus the conjugation of MGB to the probe stabilizes annealing, allowing increased target discrimination, greater precision and consistence between individual assays. The probe used in the method of this invention can be also a Locked Nucleic Acid (LNA) probe which contains nucleosides having a methylene bridge that connects the 2′-oxygen of ribose with the 4′-carbon of the ribose ring. The LNA probe is also labeled with a fluorescent dye at 5′ portion and quenched with a black hole quencher at its 3′ portion. The LNA fluorescent probe may also increase thermal stability and hybridization specificity. Two or more gene markers can be multiplexed in a single amplification reaction to save time and reagents. A housekeeping gene such as beta-actin can be used as the internal reference. A real-time quantitative PCR is carried out to detect methylation-specific amplification reaction of the gene markers. The commercially available fully methylated DNA can be used as a positive control. Because of the high ratio of unmethylated background DNA simultaneously isolated from skin samples, measurement of unmethylated DNA amplification of the gene markers may not be necessary. A methylation index (MI) for each gene marker or total MI for the panel of gene markers assayed can be calculated based on the CT value of amplification reaction, according to the following equation:

$$Methylation index ( M I ) = \frac{{2}^{- ( G sample CT \text{-} G control CT )}}{{2}^{- ( R sample CT \text{-} R control CT )}} \times 100 \%$$

Here, G represents genes of interest and R represents the internal reference. Number of MI may vary, depending on the ratio of methylated DNA, from 0 to thousands. Based on the degree of MI, number of the methylated gene markers, skin quality and/or beauty indicator status can be determined. The cutoff points for discriminating among different levels of each indicator could be established through the test of large number of skin samples.

According to the method of this invention, more preferably the method for skin quality and/or beauty indicators-associated gene methylation/demethylation determination is related to the targeted bisulfite sequencing. For targeted bisulfite sequencing identification of DNA methylation status of each gene marker-associated with skin quality and/or beauty indicators, the collected sample DNA is bisulfite modified and bisulfite converted DNA is then amplified with a set of primers targeting the CpG-rich regions in the promoter or exon 1 of a gene marker. The PCR amplification method is known to those of ordinary skill in the art. In general, the PCR reactions can be set up by adding the sample, dNTPs, and appropriate polymerase such as hotstart polymerase, primers and buffer. The PCR is performed for a number of cycles, for example 40 cycles with a defined temperature and time length for each cycle. The primer sequences are complementary to the sequences of sample DNA converted with bisulfate. Such sequences of sample DNA do not contain CpGs and are able to initiate synthesis of a primer extension product for both methylated and unmethylated DNA sequences. The multiplexed PCR can also be carried out so that more than one target sequence can be amplified by using multiple primer pairs in a reaction mixture. And such multiplexed PCR can save time and need much less amount of sample. To have a successful multiplexing reaction, the primer length will be designed in a range of 18-22 bps and Tm is between in the 60-65° C. The primer pair should result in an amplicon size between 100 and 180 bps. The primer dimmers and cross-complementarity should be avoided. As soon as the amplification is completed, the amplicons are purified. The column-based or magnetic beads-based PCR amplicon kits can be available on market. These kits include but are not limited to QIAquick PCR Purification Kit from Qiagen, EpiNext DNA Purification HT System from Epigentek, GeneJET PCR Purification Kit from Fisher, and many other suppliers.

According to the method of this invention, the purified amplicons are used for DNA library construction. The amplicons are repaired by an end repair reaction using methods or kits such as NEB End Repair kit so that the fragment end is blunt ended. Single nucleotide ‘A’ can be then added to the blunt ended 3′ terminus of each strand of the target amplicon duplexes by a reaction with Taq or Klenow exo-polymerase. The amplicons are then ligated to adaptors. The adaptors consist of two oligonucleotides and can be partially complementary to be able to form a region of double stranded sequence after annealing. The sequence length of the adaptors can be from 10 nucleotides to 100 nucleotides, preferably from 20 to 60 nucleotides, more preferably from 30-40 nucleotides. The adaptors are ligated to the both 5′ and 3′ end of the target amplicons to form adaptor-target constructs. The ligation reaction can be performed by incubating the adaptors and amplicons with ligation enzymes such as T4 DNA ligase. The nucleotide sequence of the adaptors is generally not limited to the invention and may be selected by the user. However the sequences of the individual strands in the non-complimentary region of the adaptors should not exhibit any internal self-complementarity as it could lead to self-annealing or formation of hairpin structures under standard annealing conditions. The adaptor could have a biotin molecule at the 5′ end to enable solid-phase capture of the adaptor-target constructs, for example, onto streptavidin magnetic beads or plates. The adaptor may also include “tag” sequences to mark template molecules derived from a particular source or include non-natural nucleotides.

The ligated samples are purified and size selected to remove unbound adaptor molecules. Any suitable methods can be used to remove excess unbound adaptors. For example, using PCR purification column from Qiagen could help to eliminate unbound adaptors from the samples and running the column-purified samples on 2% certified low range ultra agarose gel can help to select the desired fragment size. The beads-based DNA purification including EpiNext DNA Purification HT System (Epigentek) or Agencourt AMPure method (Backman Coulter) is also helpful to remove unbound adaptors. The desired fragment size is from 100-600 bps, preferably 150-400 bps, and more preferably 200-300 bps.

Any PCR methods can be used for amplifying the ligated samples. These methods are known to those of ordinary skill in the art. In general, the PCR reactions can be set up by adding sample, dNTPs, and appropriate polymerase such as Pfu Turbo polymerase, primers and buffer. The PCR is performed for a low number of cycles, for example 15 cycles with a defined temperature and time length for each cycle. The primers (ex: Primer A and primer B) used for the PCR reaction should be able to anneal to each individual strand of the adaptors ligated at 5′ and 3′end of the adaptor-target-adaptor constructs and is able to be extended in order to generate one complementary to each strand of the adaptor-target-adaptor polynucleotide. Based on the methods of this invention, the unligated DNA fragments will not be amplified even if a few of these fragments are in the samples. Thus only adaptor-nucleic acid constructs with intact adaptors on the both ends can be amplified.

The library produced by the PCR amplification can be further purified with various PCR purification methods. These methods including PCR purification kits (Qiagen) are known to those of ordinary skill in the art. The purified library can be further validated by measuring size, concentration and sequence of the library. The size of the library can be determined by running the library on 2% agarose gel to check whether the size range is as expected. The size of the library can also be obtained by bioanalyzer analysis. The concentration of the library can be obtained by measuring its absorbance at 260 nm.

In an embodiment of the invention, the constructed DNA library is used for deep sequencing analysis. The sequencing is conducted according to the procedure recommended for the Illumina. The indexed library DNA from each sample can be pooled and denatured. Phix is added into denatured amplicon library at a desired concentration as a control. A sample sheet containing sample names and corresponding index information can be generated and specified to generate FASTQ files. The sample is added into the reagent cartridge in the corresponding well of Illumina sequencing system. These systems include but are not limited to iSeq-100, miniSeq, MiSeq, Hiseq-2000, HiSeq-2500, HiSeq-4000, Nextseq-500, and NovaSeq-6000. Sequencing reactions can be run on one of these systems. As soon as the sequencing reaction is completed, the sample index barcode for every read will be determined and each lane of BCL files will be demultiplexed. BCL files will be converted to FASTQ files. FASTQ reads are mapped and alignment data from aligned BAM files will be merged with molecular index data from unaligned BAM files.

As soon as FASTQ files are generated, bioinformatics analysis of FASTQ files can be performed to identity differentially methylated cytosines (DMC) or regions (DMR). The ratio of the methylated (unconverted) cytosine at CpG sites to the total CpG sites of the detected DNA sequences is then calculated. An epigenetic skin profile can be generated for each sample based on the methylation score for each gene which is calculated from the ratio of the unconverted cytosine at CpG sites to the total CpG sites of the detected DNA sequences. The methylation score can be graded as 0%-100% and preferably as 0-1 and then each skin quality/beauty indicator function scale can be correlated with total methylation scores of each gene panel corresponding to each skin quality/beauty indicator.

### Example 1

The experiment was carried out to isolate DNA from skin samples of volunteers.

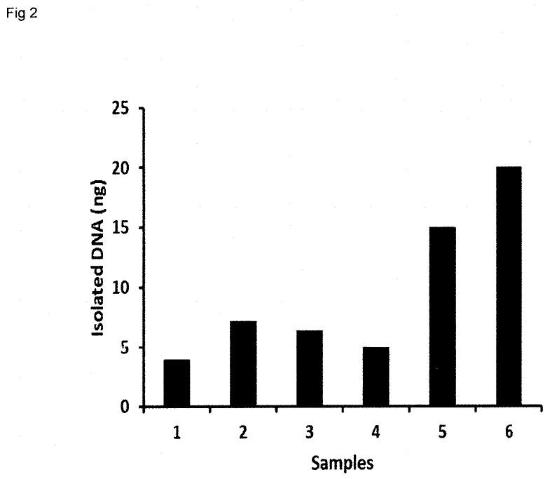

The tape discs (D-squame, Cuderm Corpotation) were placed on 4 areas: back of hand, left and right; behind the ear, left and right. One area was applied each time. The tape discs were held and pressed for approximately 2 seconds each time and then detached for a total of 5 times. The tape discs were put into a 60 mm plate, 250 ul of DNA lysis buffer were added cover the sample surface for 5 min, and then the sample surface was washed for 7-8 times by pipetting in and out the lysis buffer. The lysis buffer containing the sample is collected and put into a 0.5 ml of PCR vial, 20 ul of proteinase K solution (10 mg/ml) is added, label the sample and incubate at 60° C. for 30 min. DNA was purified with use of the EpiNext DNA purification HT system (Epigentek) and DNA was eluted to a 0.2 ml PCR tube for bisulfite conversion. DNA concentration was measured with a fluorescence DNA quantification kit (Epigentek). As shown in the FIG. 2, 4-20 ng of DNA can be isolated from each of 6 volunteers.

### Example 2

The experiment was carried out to generate PCR amplicons with bisulfte converted DNA.

Genomic DNA isolated from skin samples. DNA was treated with bisulfite reagents with use of the Methylamp DNA Modification Kit (Epigentek). 10 μl of DNA sample (5-20 ng) were used for bisulfite conversion. The reaction tubes were placed in a thermal cycler with heated lid and run with the following programs: 4 min at 95° C., 60 min at 65° C., 1 min at 95° C., 30 min at 65° C. The bisulfite treated DNA was purified by adding the sample solution into a spin column. DNA bound on the column was treated with 100 μl of alkali desulphonation solution and then washed with 200 μl of 90% ethanol for twice by centrifugation at 12,000 rpm for 1 min. The DNA was then eluted in 10 μl of Tris buffer.

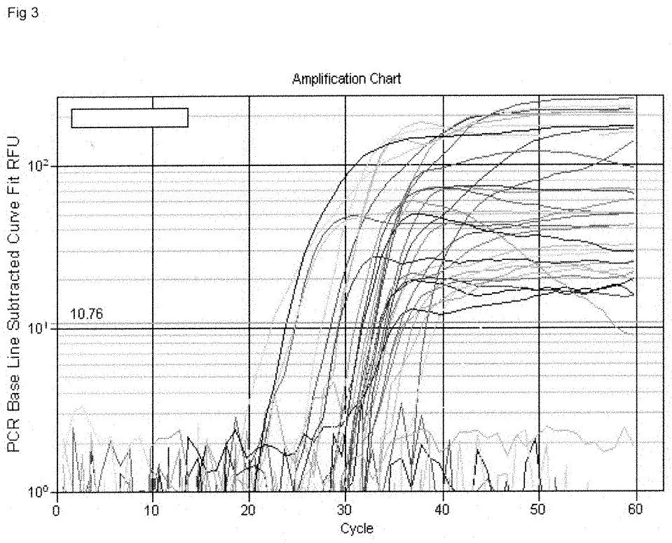

The bisulfite-treated samples were amplified by PCR reactions. (a) The real time PCR reaction was set up as follows: DNA sample 1 μl, 2×SYBR green PCR master mix (Epigentek) 10 μl, forward PCR primers 1 μl, reverse PCR primers 1 μl. Total reaction volume was adjusted to 20 μl by adding an appropriate volume of water. The following PCR protocol was used: 7 min at 95° C., 50 cycles of 10 sec at 95° C., 10 sec at 55° C. and 12 sec at 72° C. As shown in FIG. 3, the PCR products were generated in the bisulfite-treated samples.

The generated PCR products were then purified with use of EpiNext™ DNA Purification HT System (Epigentek). In brief, add 40 ul of MQ beads to the PCR product solution and incubate at room temperature for 6 min. The beads were pulled down, wash with freshly prepared 90% ethanol and then dried. The beads were suspended in the water and PCR amplicons were eluted.

### Example 3

The experiment was carried out to construct a DNA library.

The purified PCR amplicons were quantified and then mixed in equal molar ratio. The PCR amplicon mixtures were used for DNA library construction.

End repair: The mixed amplicons were treated with a mixture of enzymes to repair, blunt and phosphorylate ends. The end-repair reaction was set up as follows: amplicons (100 ng), 10×T4 DNA ligase buffer 10 μl, dNTP mix (10 mM each) 4 ul, T4 DNA polymerase (3 U/μl) 5 ul, Klenow DNA polymerase (5 U/μl) 1 ul, T4 PNK (10 U/μ) 5 μl. Total reaction volume was adjusted to 100 μl by adding an appropriate volume of water. The reaction mix was incubated at room temperature for 30 min then purified using ethanol-acetate precipitation method and eluted in 20 μl TE buffer.

dA tailing: Repaired amplicons were incubated with Klenow exo-fragment (3′-5′ exo-) to add a single “A” base to the 3′end. The dA tailing reaction was set up as follows: DNA sample (from previous end repair step) 20 μl, 10× Klenow buffer 5 μl, dATP (2 mM) 5 μl, Klenow fragment 3′-5′exo-(5 U/μl) 3 μl. Total reaction volume was adjusted to 50 ul by adding an appropriate volume of water. The reaction mix was incubated at 37° C. for 30 min, then purified using ethanol-acetate precipitation method and eluted in 20 μl TE buffer.

Ligation: The dA-tailed amplicons were incubated with adaptors to ligate the amplicons. The adaptor 1 and the adaptor 2 were annealed at 1:1 ratio. The ligation reaction was set up as follows: DNA sample (from previous dA tailing step) 20 μl, 5 × ligation buffer 12 μl, hm adaptors (at 10:1 molar ratio to DNA sample) 8 μl, T4 DNA ligase (2000 U/μl) 5 μl. Total reaction volume was adjusted to 50 μl by adding an appropriate volume of water. The reaction mix was incubated at room temperature for 20 min. The samples were then purified with use of EpiNext™ DNA Purification HT System (Epigentek) and eluted in 12 ul of water.

Index PCR amplification: The ligated DNA samples were amplified by PCR reactions to enrich the adaptor-target-adaptor constructs. The PCR reaction was set up as follows: DNA sample 2 μl, 10 ×Pfu Turbo Cx buffer 2.5 μl, dNTPs (10 mM) 2 μl, universal PCR primer 1 μl, index PCR primer 1 μl, Pfu Tubro Cx polymerase 0.5 μl. Total reaction volume is adjusted to 25 μl by adding appropriate volume of water. The following PCR protocol was used: 2 min at 98° C. 15 cycles of 10 sec at 98° C., 90 sec at 60° C. The PCR products were purified using the EpiNext™ DNA Purification HT System. DNA was eluted in 30 μl of Tris buffer and the concentration of the eluted DNA is measured by fluorescence quantification.

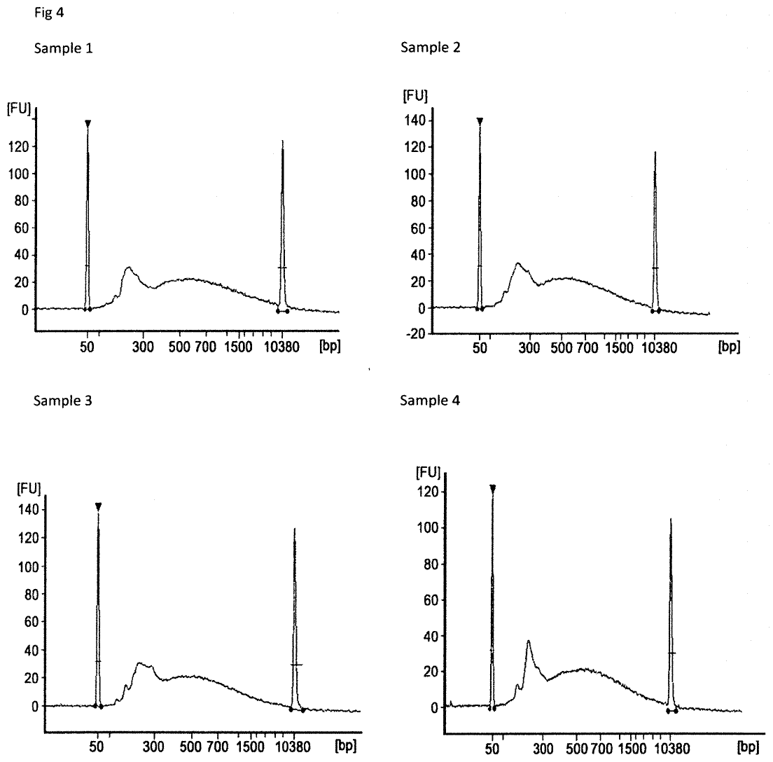

Bioanalyzer analysis of amplified constructs: DNA 7500 Lab Chips (Agilent Technologies) were loaded with samples as recommended by the manufacturer. Microchannels were filled by pipetting 9 μl of gel-dye mixture into the appropriate well and then forcing the mixture into the microchannels by applying pressure to the well via a 1-ml syringe. The ladder well and sample wells were subsequently loaded with 5 μl of marker mixture plus 1 μl of either molecular size ladder or sample, respectively. The chips were vortex-mixed for 1 min and after being vortex-mixed; chips were immediately inserted into the Bioanalyzer 2100 and processed. The results were as shown in the FIG. 4.

### Example 4

The experiment was carried for library DNA sequencing and bioinformatics analysis of sequencing data.

Amplicon library sequencing: The library DNA from each sample was pooled and denatured with 0.2 M NaOH. The denatured DNA was diluted to 10 pM with hybridization buffer (Illumina). Phix control at final concentration of 5% was added into denatured amplicon library. A sample sheet containing sample names and corresponding index information is generated and specified to generate FASTQ files only. Total of 600 μl diluted and denatured library were added into the reagent cartridge in the corresponding well of Illumina Hiseq 4000 and the sequencing reactions were run to completion. The sample index barcode for every read was determined; each lane of Illumina BCL files was demultiplexed and BCL files were converted to FASTQ files. FASTQ reads were mapped and alignment data from aligned BAM files was merged with molecular index data from unaligned BAM files.

Bioinformatics data analysis: Quality control is first performed on the Illumina raw reads using FASTQC, version 0.10.1. Low-quality read removal, trimming of the 3′ Illumina adapter, and removal of trimmed reads shorter than 20 bp were performed on the raw reads using Trin Galore. Trimmed reads are mapped to the UCSC Homo sapiens (human) genome sequence (version hg19) using a methylation-aware mapper, bismark, version 0.13.0.

Bismark stores bisulfite read mapping and methylation calling in the SAM format. For each sample, a summary report in HTML is generated, which includes alignment and cytosine methylation statistics. Samtools, version 0.1.19-96b5f2294a, is utilized to sort the SAM file produced by bismark and remove the duplicate reads due to PCR amplification. Methylation information is extracted from the final bismark mapping result at the base resolution. Minimal read coverage of 5 and minimal quality score of 20 at each base position are applied.

Methylation QC: The pipeline starts with the methylation information extracted from bismark mapping results in the CpG context. For each sample, histograms of CpG methylation percentage and coverage are generated for sample-level quality control. The resulting CpG sites are filtered based on coverage, and merged for comparative analysis. Only those CpG sites that are covered in all replicates are merged. Sample correlation is performed and presented in a scatter plot with correlation coefficients, a dendrogram.

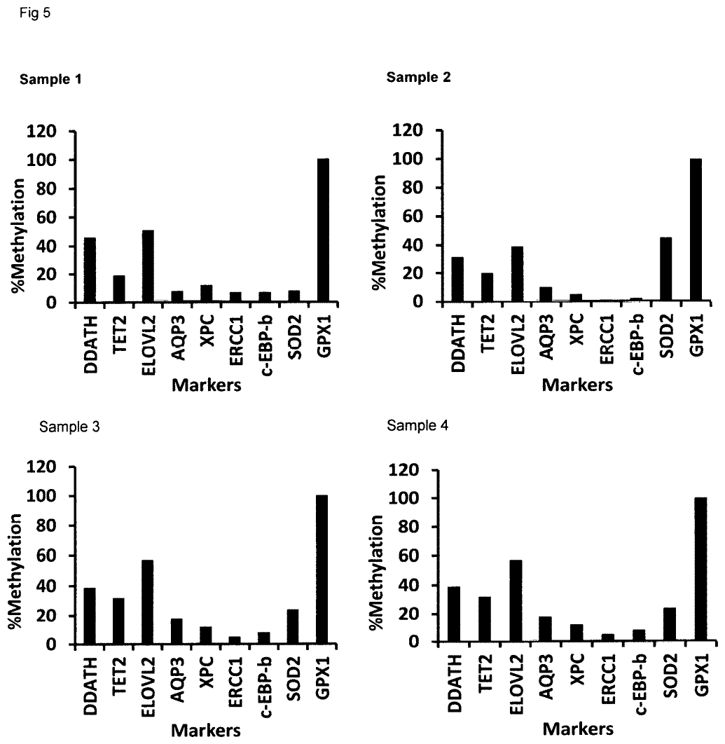

DMC and DMR analysis: DMC analysis is performed in the CpG context. Samples are filtered by coverage, normalized, merged, and subjected to DMC identification. The identified DMCs are annotated against the human hg19 RefSeq genes, and the hg19 CpG islands/shores. DMR analysis is performed in the CpG context using tiling windows, and CpG islands. Read counts with methylated cytosines (unconverted cytosines) and un-methylated cytosines (converted cytosines) in each region are summed up. The coverage for the region is calculated as (#methylated cytosines+#un-methylated cytosines) and percentage of methylation for the region is calculated as (#methylated cytosines)/(#methylated cytosines+#un-methylated cytosines)×100. Each region is then subject to coverage filtering, normalization, and differential analysis. Tiling windows and CpG islands-based DMRs are annotated against the hg19 RefSeq genes. The percentage of methylation of several genes as example was shown in FIG. 5.

### Example 5

Correlation of epigenetic score and function scale skin quality and/or beauty indicators.

Methylation score for each gene was calculated based on the ratio of the unconverted cytosine at CpG sites to the total CpG sites of the detected DNA sequences. The total methylation score for each skin quality and/or beauty indicator-associated gene panel was obtained by summing a methylation score of each gene of the panel and

What we claim is:

1. A method for generating an epigenetic skin profile associated with skin quality and/or beauty comprising: a) collecting a biological sample from a skin site in a non-invasive manner; b) determining epigenetic status of the said biological samples with a appropriate method; and c) identifying and analyzing the epigentic markers that are associated with skin quality and/or beauty, thereby generating the individualized epigenetic profile that reveals and gives an overall rating of the current status of one or more indicators of skin quality and/or beauty.

2. The method according to claim 1 wherein the said epigenetic status is referred to DNA methylation.

3. The method according to claim 1 wherein the skin quality and/or beauty comprise one or more of the following indicators: aging, firmness and elasticity, moisture, DNA damage protection, skin cell renewal/regeneration capacity, antioxidant protection capacity, sensitivity and inflammatory control and skin pigmentation.

4. The method according to claim 1 wherein the biological sample is cells or DNA.

5. The method of claim 1 wherein the non-invasive manner comprises lifttape, adhesive sheet and cotton tip swab.

6. The method of claim 1 wherein the appropriate method comprises methylation specific PCR, microarray on a chip and DNA sequencing.

7. The method of claim 1 wherein the overall rating comprises a rating system of different function grade for one or more indicators of skin quality and/or beauty.

8. A method of determining epigenetic status associated with skin quality and/or beauty comprising: a) collecting a sample from one or multiple skin sites in a non-invasive manner; b) identifying the DNA methylation markers that are indicative of one or more indicators of skin quality and/or beauty and analyzing the marker methylation percentage with an appropriate analytic assay.

9. The method of claim 8 wherein the said sample sites comprise face, ear, neck and hand.

10. The method of claim 8 wherein the said DNA methylation markers indicative of aging and elasticity, mapped with two or more genes selected from the group comprise: TOM1L1, NPTX2, ADAR, ITGA2B, PDE4C, CLCN6, TBX20, STAT5A, WT1, ASPA, KCNQ1DN, TRIM58, GRIA2, SEC3112, DDAH2, TET1, TET2, ELOVL2, p16INK4a, BIRC4BP, ITGA2B, EDARADD, MMP1, MMP2, MMP3, MMP-8, MMP-9, MMP-13, SEC31L2, B3GALT5, RAGE, STXBP5L, COL1A1, COL1A2, COL3A1, COL4A1, COL5A1, TIMP-1, TIMP-2, P-63, Satb1, ELN, ACE, FOXO3, and HIF1A.

11. The method of claim 8 wherein the said DNA methylation markers indicative of moisture and DNA repair, mapped with two or more genes selected from the group comprise: AQP-1, AQP-3, AQP5, AQP11, INV, FLG, Brg1, MBD4, MBD2, TDG, OGG1, MUTYH, NEIL1, NEIL2, NEIL3, MGMP, MSH2, MSH3, MSH6, MLH1, MLH3, XPA, XPC, RAD23A, RAD23b, DDB1, DDB2, CETN2, RPA1, RPA2, RPA3, ERCC1, ERCC2, ERCC3, ERCC4, ERCC5, ERCC6, ERCC8, GTF2H1, GTF2H2, GTF2H3, GTF2H4, GTF2H6, CDK7, CCNH, MNAT1, LIG1, UVSSA, XAB2, MMS19, RAD51, RAD51B, RAD51D, XRCC2, XRCC3, XRCC4, XRCC5, XRCC6, RAD50, and RAD52,

12. The method of claim 8 wherein the said DNA methylation markers indicative of cell regeneration/differentiation and sensitivity response, mapped with two or more genes selected from the group comprise: DDAH2, c-EBP-beta, p63, β1 integrin, K14, KMT2D, IGF-1, Dnmt1, Fra-2/AP-1, P16, P21, P27, TLR3, S1007A, Survivin, OVOL2, SOX2, CBX4, K1, K6, K10, K16, k17, CTGF, FLG, TNF-a, IL-6, SOCS1, TNFAIP3, IL-8, NF-kb, IL-1a, IL-1b, TNIP1, IL12B, IL13, CARD11, CCL5, IL-17, CCL2, INF-α, INF-β, INF-γ, IL-4, IL-5, IL-18, IL-10, P38, ERK, JNK, MAP3K, CREB, SRF, TRAF3, and TLR2.

13. The method of claim 8 wherein the said DNA methylation markers indicative of anti-oxidation and skin pigmentation, mapped with two or more genes selected from the group comprise: NRF2, NF-kappaB, FOXM1, NQO1, SOD2, GPX1, GPX2, GPX4, Catalase, GCLC, NADPH, TXN, G6PD, HMOX1, TXNRD1, PRDX3, PRDX4, GSTP1, SOD1, GCLM, GBA, SMPD1, HO-1, KITL, TYR, TYRP1, KRT75, SLC24A5, SLC45A2, IRF4, MC1R, ASIP, POMC, OCA2, MITF, RAB27A, and MLPH.

14. The method of claim 8 wherein the said the marker methylation percentage is the ratio of the unconverted cytosine at CpG sites to the total CpG sites of the detected DNA sequences for each panel of gene markers corresponding to one or more indicators of the skin quality and/or beauty.

15. The method of claim 8 wherein the said the analytic assay is one or more of the methods comprise: MS-PCR and sequencing, pyrosequencing, reduced representation bisulfite sequencing (RRBS), targeted bisulfite sequencing

16. A method of generating an individualized epigenetic profile that reveals and gives an overall rating of the current status of one or more indicators of skin quality and/or beauty comprising: a) performing a DNA methylation test on a biological sample taken from the individual's skin to identify methylation markers of the skin sample; b) obtaining a epigenetic score of each indicator of the skin quality and/or beauty based on the methylation percentage of gene markers associated with each indicator of the skin quality and/or beauty; c) determining the overall rating of the current status of one or more indicators of the skin quality and/or beauty with a rating system of different function grade for one or more indicators of skin quality and/or beauty.

17. The method of claim 16 wherein the said DNA methylation test is to detect a group of DNA methylation markers associated with one or more indicators of skin quality and/or beauty with one of the methods comprise: MS-PCR and sequencing, pyrosequencing, reduced representation bisulfite sequencing (RRBS), targeted bisulfite sequencing.

18. The method of claim 16 wherein the said epigenetic skin score is calculated based on the methylation percentage of each panel of gene markers associated with one or more indicators of the skin quality and/or beauty and graded as 0-1 or 0-100% in the scale.

19. The method of claim 16 wherein the said rating system is based on total methylation scores of each gene panel corresponding to each skin quality/beauty indicator and consist of normal, minor decrease, moderate decrease, high decrease and loss of function of each indicator of skin quality and/or beauty.
